(19) United States
(12) Patent Application Publication
(10) Pub. No.: US 2025/0376673 A1
(43) Pub. Date: Dec. 11, 2025

(54) METHOD AND SYSTEM FOR RAPID ISOLATION OF NUCLEIC ACIDS DIRECTLY FROM SAMPLES OF WHOLE BLOOD

(71) Applicant: IST Innuscreen GmbH, Berlin (DE)

(72) Inventors: Timo HILLEBRAND, Hoppegarten (DE); Kristin Wessel, Buergel (DE)

(73) Assignee: IST Innuscreen GmbH, Berlin (DE)

(21) Appl. No.: 18/260,188

(22) PCT Filed: Dec. 30, 2021
(86) PCT No.: PCT/EP2021/087873

§ 371 Date: Jan. 19, 2024

(30) Foreign Application Priority Data
Dec. 30, 2020 (DE) 10 2020 135 124.0

(51) Int. Cl. C12N 15/10

(52) U.S. Cl. CPC C12N 15/1013

(57) ABSTRACT

A method is developed for isolating nucleic acids from biological cells. After lysis of the cells, the nucleic acids contained in the cells are bound to a solid phase and treated with a wash buffer containing at least a lithium salt. A similar system is developed where the wash buffer includes a buffer known per se for lysis of biological cells; a buffer known per se for binding the nucleic acids from the lysed cells to a solid phase; a wash buffer containing at least a lithium salt; further wash buffers known per se; buffers for eluting the washed nucleic acids; and at least one solid phase comprising preferably a plastic material having a rough surface wherein said roughness of the surface is recognizable by looking at it or touching it and said rough surface can be immersed in the buffers mentioned.

The invention relates to the use of a commercially available generation of devices by means of with which normally nucleic acids are automatically purified from a biological sample, inter alia directly from whole blood, with the aid of magnetic or paramagnetic particles, for purifying nucleic acids without these magnetic or paramagnetic particles.

An example of this type of devices is the so-called KingFisher Flex Magnetic Particle Processor from Thermofischer. (https://www.thermofisher.com/de/de/home/life-science/bioproduction/contaminant-and-impurity-testing/sample-prep-and-automation/kingfisher-flex-magnetic-particle-processor.html). Similar devices, which operate on exactly the same principle, are also available from other suppliers. All these devices operate according to the following principle:

These devices use plastic combs into which magnetic rods are entered which then move magnetic particles in a walk-away process and immerse them in buffers required for a standard extraction. Extraction of nucleic acids from a blood sample begins with lysis of the sample which is located in a cavity of a 96-well deep well plate together with a lysis buffer for example, and, if necessary, a proteolytic enzyme.

After lysis is complete, a binding buffer or alcohol and magnetic or paramagnetic particles are usually added to the lysate For some providers the blood sample to be processed can contain a binding buffer and magnetic or paramagnetic particles also already during lysis. These then no longer need to be added after lysis. Essential for the extraction in all these methods is the use of the magnetic or paramagnetic particles, since the nucleic acids are bound to the particles after their release. The so-called plastic combs then move according to a predefined programming into further cavities, in which the reagents necessary for the extraction are located. The particles with the bound nucleic acids are then also moved via this progressive movement from cavity to cavity. This is done by entering a magnetic rod into the plastic combs so that the particles can then be collected on the outside of the plastic combs and transported from cavity to cavity. By means of this simple automation method, the extraction of nucleic acids is carried out according to the conventional principle of lysing-binding-washing-drying-eluting. Said principle is well known to the person skilled in the art. Both this type of extraction devices and the operating principle are well known and used on a worldwide basis. A general disadvantage, however, is that the use of magnetic or paramagnetic particles often leads to difficulties in the purity of the nucleic acids to be isolated. This is especially true when whole blood samples are being processed. Washing the bound nucleic acids is often difficult. In addition, the use of said particles also often results in discolored nucleic acid eluates. Another major disadvantage is that after each process step the particles have to be collected by means of the magnets which enter the combs. These steps take time and result in loss of nucleic acids if not collected completely.

In the published patent specification WO 2016/169677 A1 it is shown that by means of an automatic machine for the extraction of nucleic acids, which isolates nucleic acids by means of magnetic particles (KingFisher ml. ThermoElectron), nucleic acids can also be isolated without magnetic particles. Said device belongs to the group of automatic extraction machines described above and permits extraction of nucleic acids from a maximum of 15 samples in parallel. The publication discloses that mechanical roughening of the plastic combs alone is sufficient to bind nucleic acids to said rough surface. The publication lists some starting materials which have been used for the isolation of nucleic acids. The isolation of nucleic acids from blood is also described. The blood samples, however, were not used directly for the extraction. Only the isolation of DNA from the nucleated blood cells is described which first had to be obtained by means of preparatory steps. The disadvantages of such a method are the process steps of lysis of the erythrocytes and pelleting of the nucleated cells to be performed. Automation of these steps requires a centrifuge. It is well known that the technical implementation of a centrifuge in an automated process for the isolation of nucleic acids is complex and expensive. Use of a whole blood sample without these additional steps of pretreatment results in an extremely poor quality of the isolated DNA and clearly visible discoloration of the eluates after detachment of the DNA from the roughened plastic combs.

Such discolored eluates are also frequently observed in the extraction of nucleic acids from blood samples using magnetic particles. This is known to the person skilled in the art. The solutions used as wash buffers are not sufficiently efficient in order to produce clean nucleic acid eluates. This is even more the case if one wants to isolate nucleic acids by means of binding to rough surfaces. Therefore, it is necessary to separate the nucleated blood cells first. Thus, the process of extracting nucleic acids from blood is not a completely automated process and requires elaborate preparatory manual steps. In order to completely automate the process of nucleic acid extraction, publication WO 2018/167138 uses a different approach to solving the problem. Subject of the published patent application is a novel and greatly simplified method and agent allowing cells to be enriched from a sample and removed from the sample, then the nucleic acids contained in the cells to be released and isolated, such that the same agent used for enriching the cells is also used for isolating the nucleic acids. This means that the extraction process can be started directly with a whole blood sample. The nucleated cells are bound to the rough surface used there, subsequently lysed, the nucleic acid is released and subsequently bound to the rough material again, washed and finally the nucleic acid is eluted. Thus, the process is fully automated and provides nucleic acid of a high quality. The process, however, takes a very long time because it requires many process steps.

Use of lithium chloride for precipitation of RNA is known (https://www.thermofisher.com/de/de/home/references/ambion-tech-support/ma-isolation/general-articles/the-use-of-licl-ation-for-ma-purification.hunl). For example, the European patent specification EP 3 277 809 B1 describes the lysis of cells by means of lithium chloride at a concentration of 0.05 to 1 M. The lysis of cells by means of lithium chloride is described in the European patent specification EP 3 277 809 B1. The use of lithium salts as a component of wash buffers for purification of isolated nucleic acids is not known until now.

### Task of the Invention

The invention was therefore based on the task of eliminating the disadvantages of the technical solutions described in prior art.

### Solution of the Task

The task was solved according to the features of the patent claims.

According to the invention, a method for isolating nucleic acids—preferably directly from whole blood samples—by means of the presented generation of automatic extraction machines for magnetic particles has been provided which is significantly simpler and faster in performance than the known methods and which allows extremely high yields of nucleic acids with high quality to be isolated and which does not require magnetic particles. Another important advantage is the fact that magnetic and paramagnetic particles are often very expensive which significantly affects the price per extraction.

The invention is based on the publication WO 2016/169677 A1 which describes the isolation of nucleic acids by means of rough surfaces. Since there is no exact definition of the term “roughness”, a rough surface within the meaning of the present invention means a surface the roughness of which can be felt by looking at it or touching it. The production of a rough surface is very simple, at least in the case of plastics. All that is required is to treat the originally smooth surface with abrasive paper, a grinding device. a file or by scratching.

The present invention—in contrast to WO 2016/169677 A1 or WO 2018/167138—permits the isolation of nucleic acids directly from a whole blood sample and does not require any preparatory steps or elaborate flow protocols. There is also no need for prior binding of the blood cells to the rough surface (as described in WO 2018/167138). The invention is universal and can be carried out with all extraction devices operating according to the walk-away principle of the “KingFisher” device platform (ThermoElectron and other worldwide suppliers of devices operating according to the same principle). All of these devices operating with magnetic particles can be used for the extraction of nucleic acids without magnetic particles. This permits the extraction to be carried out much cheaper, since magnetic or paramagnetic particles are known to be expensive. The method according to the invention only requires mechanical roughening or scratching of the plastic combs which are normally used for the collection of the magnetic particles as intended. Furthermore, lysis buffers and binding buffers known to the person skilled in the art as well as proteolytic enzymes are used. Surprisingly, however, it was shown that clean nucleic acid eluates and high yields of nucleic acids are obtained if the known common wash buffer compositions were not used. These known compositions comprise alcoholic components and preferably chaotropic salts and/or Tris or EDTA additions. It turns out that said wash buffers are not suitable for isolating nucleic acids directly from a whole blood sample using rough surfaces or magnetic particles. One can observe two things when using known wash buffer compositions. Either the eluates are clean, in which case the nucleic acid yield is very low, or the eluates are discolored, in which case there is sufficient nucleic acid in the eluate, but the quality is very poor. According to the invention, a wash buffer containing lithium salt. preferably a high concentration of lithium chloride (greater than 0.1 M) and Tris, and a high concentration of ethanol, preferably above 50%, ideally fulfills the task of efficiently washing the nucleic acid bound to the roughened plastic combs. This wash buffer is used as the first wash buffer in the flow protocol. The other obligatory wash buffers are then alcoholic wash buffers with low salt concentrations and additions of Tris and/or, preferably 80%. pure ethanol. The decisive factor is the use of the first wash buffer according to the invention. This makes it possible to isolate nucleic acids directly from a whole blood sample with high quality and yield using the specified device systems normally operating with magnetic particles. The binding of the nucleic acids occurs solely to the plastic combs obligatory for these devices which are mechanically roughened for this purpose. This allows simple, fast and inexpensive fully automated isolation of nucleic acid from a whole blood sample. The use of the lithium wash buffer is, of course. possible in every washing step and also in the isolation of cells other than blood cells, but it is not required, only in the isolation of blood cells from whole blood. The relatively poor A₂₆₀: A₂₃₀ ratios when magnetic particles are used (see Table 1) indicate that the use of a lithium wash buffer would be reasonable in this method as well.

The method according to the invention comprises the following steps:
lysis of cells and binding of nucleic acids from these cells to a solid phase.
first washing of the nucleic acids bound to the solid phase by means of a buffer containing a lithium salt
repeated washing by means of the wash buffer or wash buffers known per se
Eluting the purified nucleic acids by means of known agents

A preferred concentration range of the lithium salt is 0.1 to 0.5 M, a concentration of 0.3 M is particularly preferred. A higher concentration than 0.5 M has no measurable effect, but only increases the further washing steps.

The system according to the invention comprises:
a buffer known per se for lysis of biological cells
a buffer known per se for binding the nucleic acids from the lysed cells to a solid phase
a wash buffer containing at least a lithium salt
further wash buffers known per se
agents for eluting the washed nucleic acids
at least one solid phase comprising at least one plastic material having a rough surface wherein said rough surface is immersed in the buffers mentioned.

Any type of polymer can be used as a plastic material including those produced in a 3D printing process. In the case of the latter, roughening is unnecessary (because the method of layer-by-laver production produces a roughness that is already required but it is currently uneconomical.

An automated procedure is also an object of the invention using devices performing automated isolation and purification of nucleic acids by means of magnetic separation. By not using magnetic particles by simply roughening the surface of the plastic combs, the automated procedure is much simpler.

The invention will be explained in more detail below by means of an embodiment. The embodiment, however, does not represent a limitation of the invention.

### Embodiment

Isolation of DNA from 4 different whole blood samples using the agents according to the invention and a conventional kit for isolation of DNA from whole blood by means of magnetic particles.

The extraction was performed using the KingFisher Flex magnetic particle processor (ThermoElectron). For this purpose, the plastic combs, into which the magnetic rods enter in the standard process, were used for a purpose other than intended and serve to bind and subsequently extract the nucleic acid. According to the present invention, the plastic combs were roughened using a grinding device. The wash buffer used for the first washing step was the wash buffer according to the invention (50 mM Tris HCl; pH 7.5, 0.3 M lithium chloride, 80% ethanol). The deep well plates were loaded with the required buffers as follows:
plate 1 (lysis/binding): 200 μl whole blood, 300 μl lysis buffer CLS and 20 μl proteinase K
plate 2 (washing 1): 50 mM Tris HCl; pH7.5, 0.3 M lithium chloride, 80% ethanol)
plate 3 (washing 2): 20 mM Tris HCl; pH7.5. 10 mM NaCl, 80% ethanol)
plate 4 (washing 3): 80% ethanol
plate 5 (washing 4): 80% ethanol
plate 6 (elution): 10 mM Tris HCl; pH 8.5

The automated extraction was started. First, the sample was lysed on the KingFisher Flex. After lysis, a binding optimizer (Analytik Jena AG) and 400 μl isopropanolin were added to the first plates.

Then, the automated extraction was continued. The entire extraction process was completed after approximately 45 min.

The extraction procedure with magnetic particles was performed according to the manual. A kit from Analytik Jena (innuPREP Blood DNA Kit KFFLX) was used. The plastic material for this was not treated. The extraction process took approx. 55 minutes.

The isolated nucleic acid was detected by means of spectrophotometric measurement. In addition to the yield. the purity of the isolated nucleic acid was also determined.

TABLE 1
Results of the spectrophotometric measurement:

| | Yield | Ratio | Ratio |
|---|---|---|---|
| Sample | (μg) | A₂₆₀:A₂₈₀ | A₂₆₀:A₂₃₀ |
| blood 1/sample 1 (Li salt) | 10.9 | 1.9 | 1.7 |
| blood 1/sample 2 (Li salt) | 10.2 | 1.9 | 1.7 |
| blood 1/sample 3 (Li salt) | 10.9 | 1.9 | 1.7 |
| blood 2/sample 1 (Li salt) | 7.4 | 1.9 | 1.6 |
| blood 2/sample 2 (Li salt) | 7.5 | 1.9 | 1.6 |
| blood 2/sample 3 (Li salt) | 7.6 | 1.9 | 1.6 |
| blood 3/sample 1 (Li salt) | 7.5 | 1.9 | 1.5 |
| blood 3/sample 2 (Li salt) | 7.7 | 1.9 | 1.6 |
| blood 3/sample 3 (Li salt) | 7.8 | 1.9 | 1.5 |
| blood 4/sample 1 (Li salt) | 12. | 1.9 | 1.7 |
| blood 4/sample 2 (Li salt) | 13.3 | 1.9 | 1.7 |
| blood 4/sample 3 (Li salt) | 12.6 | 1.9 | 1.7 |
| blood 1/sample 1 (magnetic | 10.6 | 1.8 | 1.0 |
| particles) | | | |
| blood 1/sample 2 (magnetic | 10.9 | 1.8 | 0.9 |
| particles) | | | |
| blood 1/sample 3 (magnetic | 10.3 | 1.8 | 0.9 |
| particles) | | | |
| blood 2/sample 1 (magnetic | 7.7 | 1.8 | 0.9 |
| particle) | | | |
| blood 2/sample 2 (magnetic | 7.2 | 1.8 | 0.9 |
| particles) | | | |
| blood 2/sample 3 (magnetic | 7.4 | 1.8 | 0.8 |
| particles) | | | |
| blood 3/sample 1 (magnetic | 7.7 | 1.8 | 0.9 |
| particles) | | | |
| blood 3/sample 2 (magnetic | 7.3 | 1.8 | 1.0 |
| particles) | | | |
| blood 3/sample 3 (magnetic | 7.9 | 1.8 | 1.0 |
| particles) | | | |
| blood 4/sample 1 (magnetic | 11.5 | 1.8 | 1.2 |
| particles) | | | |
| blood 4/sample 2 (magnetic | 11.8 | 1.8 | 1.1 |
| particle) | | | |
| blood 4/sample 3 (magnetic | 12.4 | 1.8 | 1.2 |
| particles) | | | |

As the results impressively show, DNA can be isolated directly from whole blood samples using the agents according to the invention and the magnetic particle processor device. The yields are comparable to those of a conventional extraction method using magnetic particles. It is impressively shown, however, that the well known poor ratios A₂₆₀:A230, when magnetic particles are used, do not occur with the agents according to the invention. In contrast to the conventional method, the ratios are significantly better. No eluates are discolored. With the conventional method, the eluates were clearly discolored. With the invention, the extraction can be performed faster than with the conventional method. The method is also much less complex, since all the steps for collecting and transporting the magnetic particles are not required. Finally, the method is cheaper since it is carried out without magnetic particles.

1. A method for isolating nucleic acids from biological cells, comprising:
a) lysing of cells and binding of nucleic acids from these cells to a solid phase; and
b) washing of the nucleic acids bound to the solid phase with a wash buffer containing a lithium salt.

2. The method according to claim 1, wherein a concentration of the lithium salt is between 0.1 and 0.5 M.

3. The method according to claim 1, characterized by further comprising:
c) repeated washing by means of the wash buffer or wash buffers known per se; and
d) eluting the purified nucleic acids by means of known buffers.

4. The method according to claim 1, wherein roughened plastic materials serve as the solid phase.

5. The method according to claim 4, wherein the roughened plastic materials are roughened plastic combs, which can be used for isolation of nucleic acids by means of magnetic particles in a plurality of commercially available automatic extraction machines, and a rough surface of which can be immersed in a buffer containing a lithium.

6. The method according to claim 5, wherein the several roughened plastic combs operate according to the walk-away principle.

7. A system for isolating nucleic acids from biological cells, wherein, after lysis of the cells, the nucleic acids contained in the cells are bound to a solid phase, and wherein the nucleic acids bound to the solid phase are treated with a wash buffer, the system comprising:
a) a buffer known per se for lysis of biological cells;
b) a buffer known per se for binding the nucleic acids from the lysed cells to a solid phase;
c) a wash buffer containing at least a lithium salt;
d) further wash buffers known per se;
e) buffers for eluting the washed nucleic acids; and
f) at least one solid phase.

8. The method according to claim 1, further comprising:
isolating and purifying nucleic acids from biological cells.

9. The method according to claim 8, wherein the nucleic acids are isolated and purified from whole blood.

10. A method of purifying nucleic acids, comprising:
treating nucleic acids with a wash buffer, wherein the nucleic acids are bound to a solid phase and the wash buffer contains lithium salts.

11. The method according to claim 1, wherein a concentration of the lithium salt is 0.3 M.

12. The system according to claim 7, wherein the at least one solid phase in f) comprises a plastic material having a rough surface, wherein said rough surface can be immersed in the buffers mentioned.
